(19) United States
(12) Patent Application Publication
(10) Pub. No.: US 2009/0118136 A1
(43) Pub. Date: May 7, 2009

(54) ASSAY FOR DETECTING NUCLEOTIDE SEQUENCES IN GENETICALLY MODIFIED CROPS AND PLANTS USING OPTICAL THIN-FILM BIOSENSOR CHIPS

(76) Inventor: Xing Wang Deng, Hamden, CT (US)

Correspondence Address: BACHMAN & LAPOINTE, P.C., 900 CHAPEL STREET, SUITE 1201, NEW HAVEN, CT, 06510, US

(21) Appl. No.: 12/344,974

(22) Filed: Dec. 29, 2008

Related U.S. Application Data
(63) Continuation of application No. PCT/US2007/072306, filed on Jun. 28, 2007.
(60) Provisional application No. 60/817,136, filed on Jun. 28, 2006.

(51) Int. Cl. C40B 30/04; C12Q 1/68

(52) U.S. Cl. 506/9; 435/6

(57) ABSTRACT

A process for detecting at least one DNA sequence using an optical thin-film biosensor chip includes the steps of placing a sample to be tested in contact with at least one capture probe attached to an optical thin-film biosensor chip; incubating the sample in the presence of an enzyme and a substrate; identifying a change in the color of the sample; and detecting the sample comprises at least one DNA sequence.

### CROSS-REFERENCE TO RELATED APPLICATIONS

This non-provisional patent application claims priority to U.S. Provisional Application No. 60/817,136 entitled “A Simple and Reliable Assay for Detecting Specific Nucleotide Sequence Variations in Plants Using Optical Thin-Film Biosensor Chips”, filed on Jun. 28, 2006 in the name of Xing Wang Deng, which is incorporated by reference herein in its entirety.

### BACKGROUND OF THE INVENTION

Rapid and reliable identification of a specific nucleotide sequence in plants is desirable in essentially all aspects of plant science research and associated applications. Distinguishing between species or ecotypes or tracing patterns of inheritance at the genome level are routine laboratory procedures. In addition, customs and government inspectors, crop breeders, and commercial and food-processing sectors often need to detect genetically modified (GM) crops or microbial pathogens in plant product shipments to ensure compliance with regulatory requirements.

Presently, there are already many detection techniques available for positive identification of a specific nucleotide sequence or polymorphism. These detection assays can be classified into three broad types: polymerase chain reaction, molecular markers, and microarray techniques. The first type of assay is based on polymerase chain reaction (PCR). PCR is the most commonly used method for amplification of a specific DNA sequence. The basic technique for demonstrating the presence of an amplified DNA sequence is gel electrophoresis, a technique that allows the quantity, size, and even the sequence of the DNA to be determined (Chiueh, L. C., et al. “Study on the Detection Method of Six Varieties of Genetically Modified Maize and Processed Foods”, J. Food Drug Anal. 10, 25-33 (2002); German, M. A., et al., “A rapid method for the analysis of zygosity in transgenic plants,” Plant Sci. 164, 183-187 (2003); Gilliland, G., et al., “Analysis of cytokine mRNA and DNA: Detection and quantitation by competitive polymerase chain reaction,” Proc. Natl. Acad. Sci. USA, 87, 2725-2729 (1990); Holst-Jensen, A., et al. “PCR technology for screening and quantification of genetically modified organisms (GMOs),” Analyt. Bioanalyt. Chem. 375, 985-993 (2003); Miraglia, M., et al., “Detection and traceability of genetically modified organisms in the food production chain,” Food Chem. Toxicol. 42, 1157-1180 (2004); Rogers, H. J., et al., “Direct PCR amplification from leaf discs,” Plant Sci. 143, 183-186 (1999; Su, W., et al., “Multiplex polymerase chain reaction/membrane hybridisation assay for detection of genetically modified organisms,” J. Biotechnol. 105, 227-233 (2003); and, all of the aforementioned references are incorporated by reference herein in their entireties).

Real-time PCR is a commonly used technology for the quantification of specific DNA fragments. The amount of product synthesized during the PCR reaction is measured in real-time by detection of fluorescence signal produced as a result of amplification. Real-time PCR requires special thermal cycling machines and specific fluorescent probes. Although real-time PCR is rapid and sensitive, the process is also expensive and prone to generating false positive signals, and such misidentifications can be very costly (Baric, S., et al. “A new approach to apple proliferation detection: a highly sensitive real-time PCR assay,” J. Microbiol. Meth. 57, 135-145 (2004); Hernandez, M., et al. “Development of real-time PCR systems based on SYBR Green I, Amplifluor and TaqMan technologies for specific quantitative detection of the transgenic maize event GA21,” J. Cereal Sci. 39, 99-107 (2004); Shibata, D., et al. “Establishment of framework P1 clones for map-based cloning and genome sequencing: direct RFLP mapping of large clones,” Gene, 225, 31-38 (1998); Stubner, S, “Enumeration of 16S rDNA of Desulfotomaculum lineage 1 in rice field soil by real-time PCR with SybrGreenk detection,” J. Microbiol. Meth. 50, 155-164 (2002); and, all of the aforementioned references are incorporated by reference herein in their entireties).

The second type of assay is based on molecular markers. DNA markers have now become a popular means for identification or authentication of a plant species (Andersen and Andersen, J. R., et al. “Functional markers in plants,” Trends Plant Sci. 8, 554-560 (2003), which is incorporated by reference herein in its entirety). The commonly used molecular markers include restriction fragment length polymorphism (RFLP), random amplified polymorphic DNA (RAPD), simple sequence repeats (SSR), and single nucleotide polymorphisms (SNPs). RFLP is based on hybridization of cloned DNA to enzymatically digested DNA fragments from a test sample (Bai, S. L., et al. “Mapping the srs-1 gene of split rice spikelet and analysing it's homeotic function,” Sci. China (Ser. C), 43, 369-375 (2000); Cavallotti, A., et al. “New sources of cytoplasmic diversity in the Italian population of Olea europaea L. as revealed by RFLP analysis of mitochondrial DNA: characterization of the cox3 locus and possible relationship with cytoplasmic male sterility,” Plant Sci. 164, 241-252 (2003); Yu, Y., et al. “Interval mapping of quantitative trait loci by molecular markers in rice (Oryza sativa L.),” Science in China (Ser. B), 38, 422-428 (1995); Zhang, G.-Y., Guo, et al. “RFLP tagging of a salt tolerance gene in rice,” Plant Sci. 110, 227-234 (1995); and, all of the aforementioned references are incorporated by reference herein in their entireties). It involves restriction enzyme digestion, gel electrophoresis, transfer to the membrane, and hybridization to a labeled DNA probe. This method is accurate but lengthy and labor-intensive.

The next type of molecular marker is RAPD. RAPD is based on PCR amplification of random sequences in the plant genome. Extensive PCR amplification is required and the result is often contingent upon the experimental conditions for each PCR reaction (Ilbi, H., “RAPD markers assisted varietal identification and genetic purity test in pepper, Capsicum annuum,” Scientia Horticulturae, 97, 211-218 (2003); Luo, S. L., et al. “Inheritance of RAPD markers in an interspecific F1 hybrid of grape between Vitis quinquanglaris and V. vinifera,” Scientia Horticulturae, 93, 19-28 (2002); and, all of the aforementioned references are incorporated by reference herein in their entireties). SSR markers or microsatellites are unique tandem repeats interspersed throughout the genome and can be PCR amplified using primers that flank these regions (Cordeiro, G. M., et al., “Characterisation of microsatellite markers from sugarcane (Saccharum sp.), a highly polyploid species,” Plant Sci. 155, 161-168 (2000); Edwards, Y. J. K., et al. “The identification and characterization of microsatellites in the compact genome of the Japanese pufferfish, fugu rubripes: perspectives in functional and comparative genomic analyses,” J. Mol. Biol. 278, 843-854 (1998); and, all of the aforementioned references are incorporated by reference herein in their entireties). In a mixture of denatured DNA, SSR tends to reassociate quickly owing to the low complexity of the nucleotide composition. Primers for SSR analysis can be designed by consulting the GenBank for SSR loci of related species or by screening genomic libraries.

The last type of molecular marker is SNPs. SNPs are single nucleotide differences in different alleles that contribute to overall genomic diversity and natural variation in plants (Maloof, J. N., Borevitz, et al. “Natural variation in light sensitivity of Arabidopsis,” Nat. Genet. 29, 441-446 (2001), incorporated by reference herein in its entirety). The SNP-based assay can distinguish sequences that have single nucleotide differences (Lovmar, L., et al. “Quantitative evaluation by minisequencing and microarrays reveals accurate multiplexed SNP genotyping of whole genome amplified DNA,” Nucleic Acids Res. 31, e129 (2003); Rafalski, J. A, “Novel genetic mapping tools in plants: SNPs and LD-based approaches,” Plant Sci. 162, 329-333 (2002); and, all of the aforementioned references are incorporated by reference herein in their entireties). SNPs are most commonly used in the identification of distinct strains or cultivars of the same plant species. For example, on average there is a difference of one SNP every 3.3 kb in the Arabidopsis genome (a total of >37,000 SNP in 125 Mb) between the sequences of two major Arabidopsis ecotypes, Col and Ler (http://www.arabidopsis.org/cereon/). Compared to Arabidopsis, rice has possibly ten times as many SNP markers per unit DNA sequence length that distinguish between the sequenced genomes of the two major subspecies, indica 9311 (Yu, J., et al., “The Genomes of Oryza sativa: a history of duplications,” PLoS Biol. 3: e38 (2005), incorporated by reference herein in its entirety) and japonica Nipponbare (Mashima, Y., et al. “Rapid quantification of the heteroplasmy of mutant mitochondrial DNAs in Leber's hereditary optic neuropathy using the Invader technology,” Clin. Biochem. 37, 268-276 (2004), incorporated by reference herein in its entirety). A survey of the existing SNP assays and platforms reveals that the high-throughput technologies such as the Invader™ and SNiPer™ assays need expensive high-tech instrumentation to detect polymorphic differences (Mashima, Y., et al. “Rapid quantification of the heteroplasmy of mutant mitochondrial DNAs in Leber's hereditary optic neuropathy using the Invader technology,” Clin. Biochem. 37, 268-276 (2004); Pati, N., et al. “A comparison between SNaPshot, pyrosequencing, and biplex invader SNP genotyping methods: accuracy, cost, and throughput,” J. Biochem. Biophys. Methods, 60, 1-12 (2004); and, all of the aforementioned references are incorporated by reference herein in their entireties).

The third and more recently developed assay type employs microarray techniques. This type of assay relies on the capacity of a microarray to simultaneously identify a large number of specific DNA or molecular markers (Li, L., et al., “Tiling microarray analysis of rice chromosome 10 to identify the transcriptome and relate its expression to chromosomal architecture,” Genome Biol. 6, R52 (2005); Li, L., et al. “Genome-wide transcription analyses in rice using tiling microarrays,” Nat. Genet. 38, 124-129 (2006); which is incorporated by reference herein in its entirety). Selected probes are attached to a solid surface with each spot containing numerous copies of a single probe. The array is subsequently hybridized with PCR-amplified DNA labeled with a fluorescent marker that is isolated from the sample of interest. During the hybridization phase, the labeled fragments associate with probes that have complementary DNA sequences. This method has been widely applied in many research fields, but it requires expensive equipment and highly trained researchers to perform, thus limiting its general usage for breeding or routine sample identification.

Therefore, in light of the large and growing demand for detecting foreign sequences in plant products, there exists a need for an inexpensive, highly specific, and easy-to-use assay that is suited to a broad range of settings and applications.

### SUMMARY OF THE INVENTION

In accordance with one aspect of the present disclosure, a process for detecting at least one DNA sequence using an optical thin-film biosensor chip broadly comprises placing a sample to be tested in contact with at least one capture probe attached to an optical thin-film biosensor chip; incubating the sample in the presence of an enzyme and a substrate; identifying a change in the color of the sample; and detecting the sample comprises at least one DNA sequence.

In another aspect of the present disclosure, A process for detecting at least one SNP using an optical thin-film biosensor chip broadly comprises placing a sample to be tested in contact with at least one capture probe attached to an optical thin-film biosensor chip; incubating the sample in the presence of an enzyme and a substrate; identifying a change in the color of the sample; and detecting the sample comprises at least one SNP assay.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1a is a representation of a thin-film biosensor silicon chip of the prior art;

FIG. 1b is a representation of a strategy for GMO detection on thin-film biosensor chips of the prior art;

FIG. 1c is a representation of an exemplary assay strategy of the present disclosure for an SNP array using a thin film biosensor chip of the prior art;

FIG. 2a is a set of photographs showing the specificity and sensitivity of GMO detection on a chip with capture probes spotted at different concentrations;

FIG. 2b is a set of photographs showing the specificity and sensitivity of GMO detection on a chip with capture probes spotted at different concentrations.

FIG. 3a is a set of photographs showing the amplification of target DNA fragments for transgenic and endogenous genes by PCR from the seeds of each individual GMO crop;

FIG. 3b is a set of photographs showing the detection of the transgenic and endogenous genes in GMO products on thin-film biosensor chips;

FIG. 4a is a set of photographs showing the detection of SNP markers in rice on the thin-film biosensor chip;

FIG. 4b is a set of photographs showing the detection of SNP markers in Arabidopsis on the thin-film biosensor chip;

FIG. 4c is a set of photographs showing the detection of SNP markers in tomato on the thin-film biosensor chip;

FIG. 5 is a set of photographs showing the isolated and segregated mutant collection of genome-wide SNP markers from the F2 population in the top panel and a graph illustrating the correlation of signal intensity of a biosensor chip SNP assay with an abundance of specific SNP target molecules in a sample;

FIG. 6a is a diagram illustrating the mapping of a new mutation using the exemplary SNP detection assay of the present disclosure; and

FIG. 6b is a diagram illustrating the frequency of a SNP marker found on chromosome 2 conferring ecotype specificity (Col or Ler).

Like reference number and designations in the various drawings indicate like elements.

### DETAILED DESCRIPTION OF THE DISCLOSURE

The inventors of the present disclosure adapted and optimized an assay for the detection of specific nucleic acid sequences and molecular markers in plants on the surface of optical thin-film biosensor chips described in the article entitled, “Single-nucleotide polymorphism genotyping on optical thin-film biosensor chips”, by Zhong, X. B. et al., Proc. Natl. Acad. USA, 100, 11559-11564 (2003), incorporated by reference herein in its entirety. The exemplary assay described herein is also described in the article entitled, “A Simple and Reliable Assay for Detecting Specific Nucleotide Sequences in Plants Using Optical Thin-Film Biosensor Chips”, by Bai, Su-Lan et al., The Plant Journal, 49, 354-366 (2007), which is incorporated by reference herein in its entirety.

One of the advantages of this technology is that due to the optical characteristics of the thin-film biosensor chip surface, experimental results can be visualized by the unaided human eye. This technology eliminates a large initial investment in expensive instrumentation and can be widely distributed to any individual laboratory or research station having a basic molecular biology facility with PCR capability. The exemplary assay and results described herein demonstrate that the exemplary assay is rapid, robust, highly sensitive and specific, can be extensively multiplexed, and is considerably less expensive compared with the aforementioned existing technologies.

The exemplary assay described herein employs a custom-designed silicon-based optical thin-film biosensor chip to detect unique transgenes in genetically modified (GM) crops and SNP markers in model plant genomes. An aldehyde-attached sequence specific single-stranded oligonucleotide probes may be arrayed and covalently attached to a surface of the biosensor chip which is derivatized to form covalent bonds, for example, a hydrazine-derivatized biosensor chip surface. Although aldehyde is the reactive group mentioned herein, other reactive groups capable of covalently bonding to the derivatized surface of the biosensor chip may be utilized as well. Likewise, other compounds beyond hydrazine may be utilized to derivatize the surface of the biosensor chip. Unique DNA sequences (or genes) may be detected by hybridizing biotinylated PCR amplicons of the DNA sequences to probes on the chip surface. In the SNP assay, target sequences (PCR amplicons) may be hybridized in the presence of a mixture of biotinylated detector probes and a thermostable DNA ligase. Only perfect matches between the probe and target sequences, but not those with even a single nucleotide mismatch, can be covalently fixed on the chip surface. In both cases, the presence of specific target sequences is signified by a color change on the chip surface, for example, gold to blue/purple, after brief incubations with an enzyme capable of detecting the hybridized DNA sequence, such as anti-biotin IgG horseradish peroxidase (HRP), to generate a precipitable product from a substrate capable of binding with the enzyme, such as an HRP substrate. Highly sensitive and accurate identification of PCR targets can be completed within approximately 30 minutes.

Given the abundance of SNP markers spread throughout the genome, the described thin-film biosensor chip SNP assay platform can be easily adapted as an exemplary assay for a genome-wide SNP scoring system described herein. This is because the exemplary SNP assay described herein can rapidly identify target molecules with high specificity and sensitivity. With a custom-designed chip containing SNP markers that strategically span the whole genome, one can rapidly identify the genotypes of the progeny during crop breeding, as the process of progeny genotype and selection is the limiting step. Since SNPs are co-dominant markers and the exemplary assay described herein can positively identify all SNP forms at any given position in genomic DNA, adaptation of the exemplary assay described herein could significantly speed up the crop breeding process.

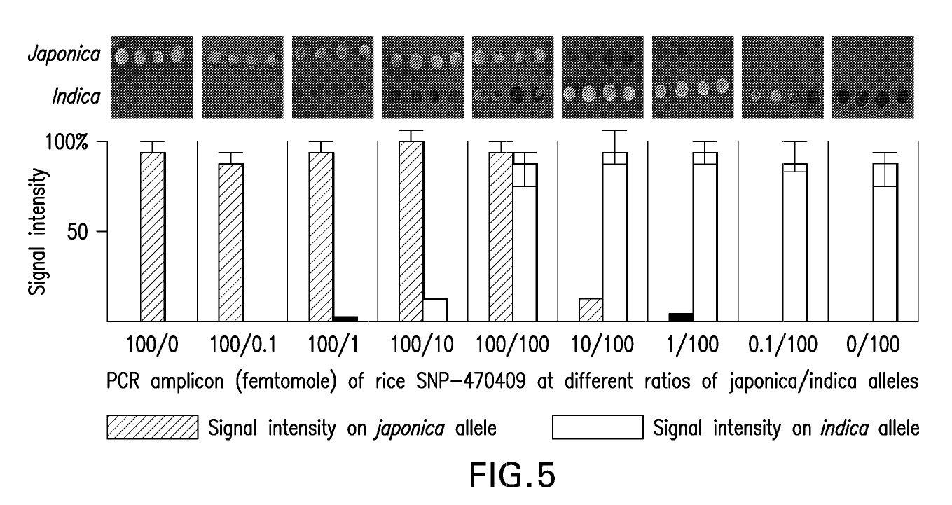

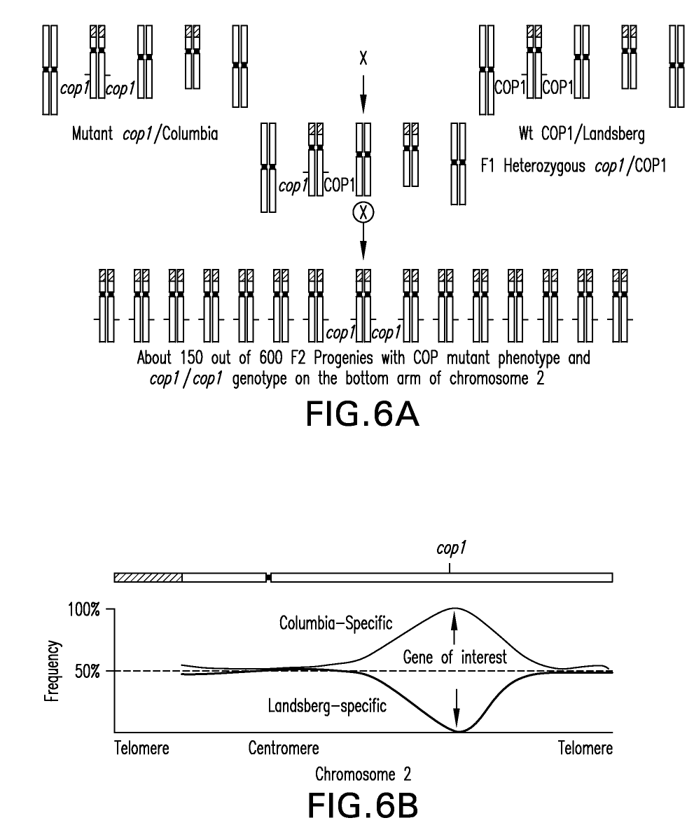

A similar custom chip covering a large number of well-spaced SNP markers of the entire genome of common research model plants may also facilitate rapid chromosomal mapping of a mutation for map-based gene cloning. Individual mutants in an F2 progeny population resulting from a mapping cross will retain the genotype of the original mutation-generating strain at the locus of the mutation. Thus, if a large number, e.g., in the hundreds, of mutants from the F2 progeny are pooled for DNA isolation and SNP marker identification, the SNP markers around the mutation locus will be biased toward the strain containing the original mutation (see FIG. 6). The SNP markers in other regions of the genome may have a relatively equal chance of having both parental types of SNP markers. As we demonstrated in FIG. 5, the exemplary SNP assay described herein may quantitatively detect the abundance of both SNP markers from a single locus in the DNA target. The exemplary SNP assay described herein may also further define one location, that is, a small genomic region, where SNP markers are biased toward the form found in the original mutant strain of the two parental strains, thus locating the mutation locus in that region.

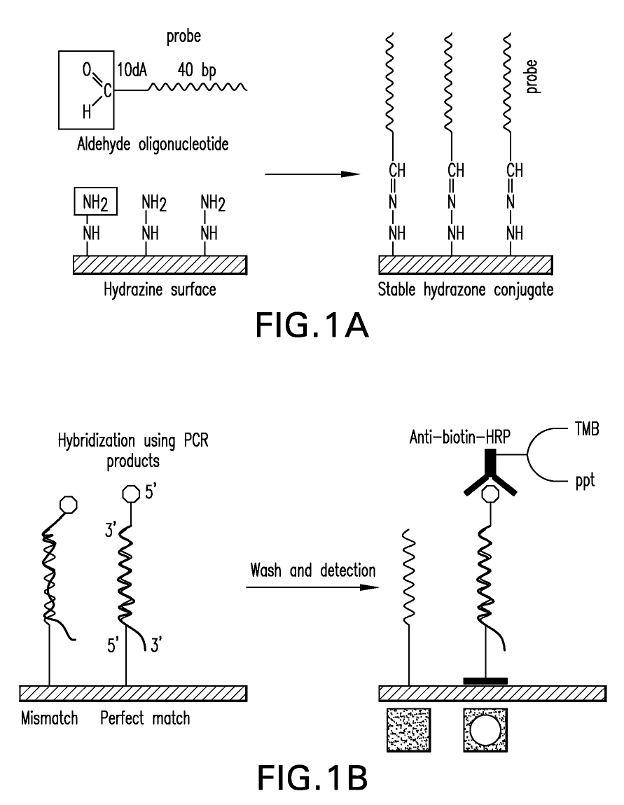

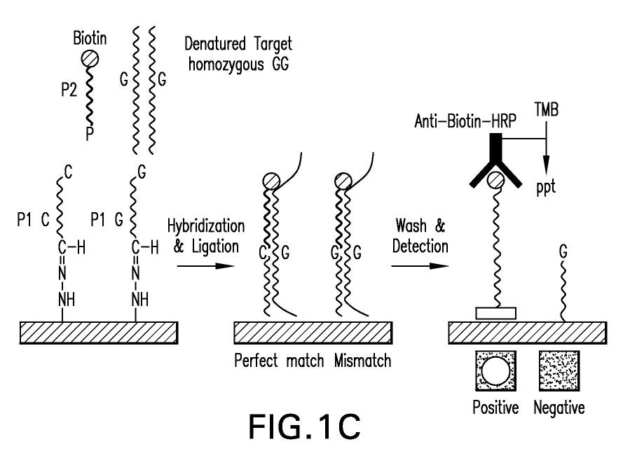

Referring now to FIGS. 1a-1c and Table 1 below, the detection of genetically modified organisms (GMO) may be accomplished by hybridizing biotinylated PCR fragments with capture probes covalently attached to thin-film biosensor silicon chips, which will be discussed in greater detail in the Experimental Section. For detection of each gene or known DNA fragment, a forward and reverse primer for each PCR reaction, and a capture probe with a forward strand sequence for spotting on the chip surface was synthesized. The reverse primer carries a covalently attached biotin at the 5′ end for usage in future detection (See Table 1 and FIG. 1b). The capture probe has an aldehyde group at the 5′ end of 10 deoxyadenosine (dA) residues, as a spacer, followed by a 40 nucleotide sequence that perfectly matches the sequence of the PCR amplicon. This capture probe may be covalently attached through its 5′ terminus to the chip surface (See Table 1 and FIG. 1A). Target DNA hybridization reactions begin with a 10 min. incubation, during which the biotinylated reverse strand of PCR amplicon binds to capture probe through standard DNA base pairing. After simple washes with 1×SSC to remove all mismatched molecules, attachment of the biotinylated PCR fragment may be detected by incubation with an anti-biotin IgG-horseradish peroxidase (HRP) conjugate and tetramethylbenzidine (TMB), a substrate that can be converted into a precipitable product by HRP to increase film thickness. This change in film thickness is due to mass deposition from the substrate and causes a distinguishable color change from gold to blue/purple on the chip surface, which can be visualized by the unaided eye or recorded by a simple digital-imaging documentation system depending on the density and size of probe spots.

TABLE 1
Oligonucleotide sequences of PCR primers and capture probes for GMO detection

| | | | |
|---|---|---|---|
| | | Fragment | |
| Primers | | length | Accession |
| and probes | Sequences | (bp) | number |

| | | | | |
|---|---|---|---|---|
| Endogenous | Lectin | F: 5′-gccctctactccacccccatcc | 118 | K00821 |
| gene | (soybean) | R: 5′-biotin-gcccatctgcaagccttmgtg | | |
| Group | | P: 5′-ALD-aaaaaaaaaacatttgggacaaagaaaccggtagcgttgccagcttcgcc | | |
| | Ivr1 | F: 5′-ccgctgtatcacaagggctggtacc | 217 | U16123 |
| | (maize) | R: 5′-biotin tgtagagcatgacgatcc | | |
| | | P: 5′-ALD-aaaaaaaaaaacactggctgcacctaccgctggccatggtgcccgatcacc | | |
| | Accg8 | F: 5′-gagaatgaggaggaccaagctc | 196 | X77576 |
| | (Canola) | R: 5′-biotin-ggcgcagcatcggctctt | | |
| | | P: 5′-ALD-aaaaaaaaaagacgaacacctattagacattcgttccattggtcgatgga | | |
| | Sadi | F: 5′-ccaaaggaggtgcctgnca | 177 | AJ132636 |
| | (cotton) | R: 5′-biotin-ttgctcatgaaatccatca | | |
| | | P: 5′-ALD-aaaaaaaaaagattgagatctttaaatctttggagggctgggctgagaac | | |
| Screening | CaMV35S | F: 5′-gctcctacaaatgccatcat | 195 | AF234316 |
| gene | Promoter | R: 5′-biotin-gatagtgggattgtgcgtca | | |
| group | | P: 5′-ALD-aaaaaaaaaacccacccacgaggagcatcgtggaaaaagaagacgttcca | | |
| | NOS | F: 5′-gaatcctgttgccggtcttg | 180 | Z29588 |
| | Terminator | R: 5′-biotin-ttatcctagtttgcgcgcta | | |
| | | P: 5′-ALD-aaaaaaaaaaatgacgttatttatgagatgggtttttatgattagagtcc | | |
| | nptII | F: 5′-ctcaccttgctcctgccgaga | 327 | U12668 |
| | | R: 5′-biotin-agtcgatgaatccagaaa | | |
| | | P: 5′-ALD-aaaaaaaaaatcgcatcgagcgagcacgtactcggatggaagccggtctt | | |
| | GUS | F: 5′-tcagcgcgaagtctttatac | 210 | U12640 |
| | | R: 5′-biotin-ttcagttcgttgttcacacaaacggtga | | |
| | | P: 5′-ALD-aaaaaaaaaacggcaaagtgtgggtcaaataatcaggaagtgatggagca | | |
| Identifying | CryIA(b) | F: 5′-atggacaacaaccacaac | 114 | AF465640 |
| gene | | R: 5′-biotin-gatgtcgatgggggtgtaa | | |
| Group | | P: 5′-ALD-aaaaaaaaaagagtgcatcccgtacaactgcctcagcaaccctgaggtcg | | |
| | CryIA(c) | F: 5′-gttcgttctcggactagttgaca | 195 | Y09787 |
| | | R: 5′-biotin-tcggcttcccactctctgaagctat | | |
| | | P: 5′-ALD-aaaaaaaaaagagcagttgatcaaccagaggatcgaagagttcgccagga | | |
| | cp4-epsps | F: 5′-gcgaagatcgaactctccg | 114 | AY125353 |
| | | R: 5′-biotin-tcaatcttaagaaactttattgcc | | |
| | | P: 5′-ALD-aaaaaaaaaaatgcctgatgagctcgaattcgagctcggtaccggatccaa | | |
| | BAR(pat) | F: 5′-actcggccgtccagtcgta | 196 | AF404854 |
| | | R: 5′-biotin-atcgtcaaccactacatc | | |
| | | P: 5′-ALD-aaaaaaaaaataggcgatgccggcgacctcgccgtccacctcggcgacga | | |

Abbreviations:
F, forward PCR primer;
R, reverse PCR primer;
P, probe;
ALD, aldehyde modification.

Referring specifically now to FIG. 1c, the biosensor chip can be further adapted to create the exemplary SNP assay described herein. To determine the presence or absence of an SNP, one pair of oligomers (P-1 capture probes) containing a 10-base space linker followed by 40 nucleotides complementary to the DNA sequence immediately adjacent to the SNP in the target gene is covalently attached to the chip surface through their 5′ termini (See for example FIG. 1a). The two sequences differ only in their 3′ terminal nucleotides which correspond to different forms of the SNP alleles of interest. A second oligonucleotide probe (P-2) contains a sequence complementary to the region adjacent to the SNP on the opposite side, as well as a biotin group at the 3′ end for detection and a phosphate group at the 5′ end to facilitate ligation. Target DNA hybridization and P-1-P-2 ligation reactions proceed simultaneously during an approximate 20 minute incubation in the presence of a thermostable DNA ligase, as hybridization with the target facilitates ligation of the P1 and P2 probes. After a stringent wash with NaOH to remove all unligated molecules, immobilized biotinylated P-2 is retained only when the P1 3′ SNP nucleotide perfectly complements its counterpart in the DNA target sequence. Ligated biotinylated P2 probe is detected by incubation with an anti-biotin IgG-HRP conjugate and a precipitable HRP substrate as described above.

### EXPERIMENTAL SECTION

The thin-film biosensor chips are commercially available from Inverness Medical-Biostar, located at Louisville, Colo. The chips were prepared by coating the surface of 10-cm-diameter silicon wafers with a 475-Å layer of silicon nitride (S₃N₄), which serves as the optical layer. To facilitate covalent attachment of biomolecules, the wafers also were spin-coated with a 135-Å layer of T structure aminoalkylpolydimethylsiloxane (TSPS) and poly(PheLys) passively adsorbed to the TSPS layer. The wafers were cut into 6×6-mm squares by using a laser knife, with each square constituting an assay chip. The ε-amino acid groups of the poly(PheLys) were converted to hydrazines to optimize the simultaneous printing of multiple oligonucleotide arrays. The poly(PheLys)-coated silicon wafers incubated in a 25-ml solution of 1-10 μM succinimidyl hydrazinium nicotinate hydrochloride, commercially available from Solulink, located in San Diego, Calif., in a 0.1 M sodium borate buffer, pH 8.1-8.4 for 2 hours followed by washing in distilled H₂O. Oligonucleotides were chemically synthesized on a standard instrument incorporating a 5′-aldehyde-substituted phosphoramidite, commercially available from Solulink, into the synthetic process.

Aldehyde-labeled oligonucleotides were diluted to different concentrations in 0.1 M sodium phosphate buffer, pH 7.8, and 1-200 nl was spotted, depending on the printing method, onto hydrazine-coated chips. After incubation at room temperature for 2 hours, the chips were rinsed with distilled H2O, and air-dried. Based on the requirements of each specific experimental application described herein, the oligonucleotide probes were specifically designed for the exemplary thin-film biosensor chip SNP arrays described herein. The oligonucleotide synthesis and design is set forth in the discussion of the experimental applications that follow.

Defining Sensitivity and Specificity Parameters of Thin-Film Biosensor Chip for Use in Exemplary Arrays

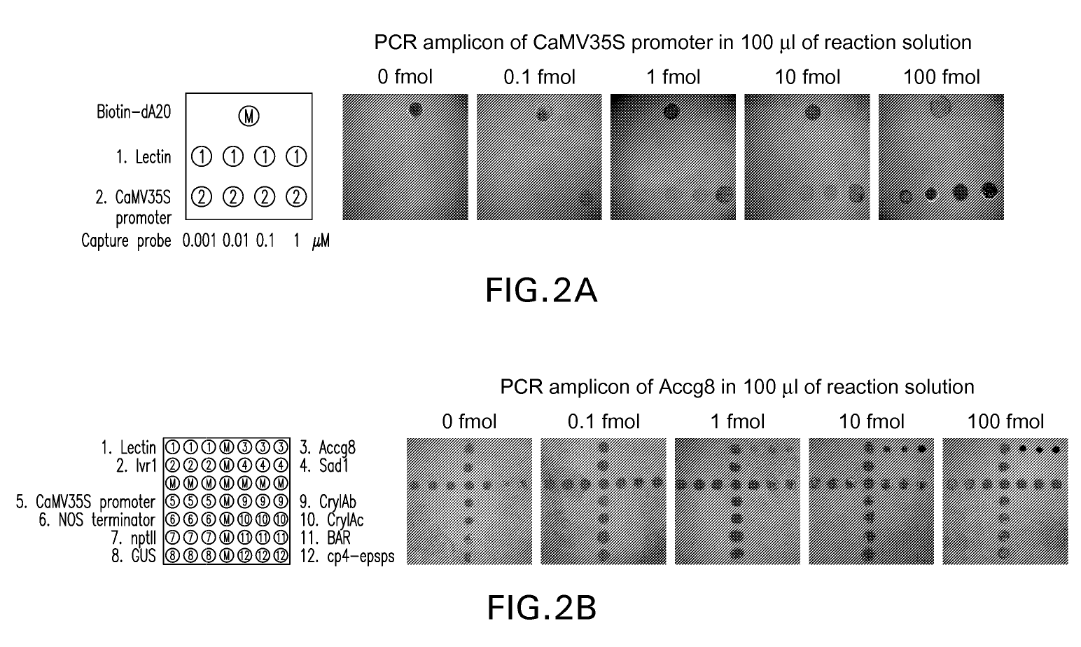

Referring now to the photographs of FIGS. 2a-2b, in order to establish the suitable probe density for spotting on a biosensor chip for use in the exemplary assay described herein, well-described lectin gene and CaMV35S promoter were selected and diluted to various concentrations. As shown in the photograph of FIG. 2a (left), batches of 200 nl of solution containing each of the two probes at concentrations of 0.001, 0.01, 0.1, and 1 μM were manually spotted on the chip surface. Identical chips were hybridized for 10 min at 42° C. with CaMV35S promoter PCR products at concentrations of 0, 0.1, 1, 10 and 100 fmol in a 100 μl total reaction volume. The chips were washed once with 0.1×SSC at room temperature, followed by a 5 minute incubation with an anti-biotin IgG-HRP conjugate and a 5 minute incubation with tetramethylbenzidine (TMB) substrate. The chips were then washed, dried, and visualized as shown in the right panels of the photographs of FIG. 2a. As expected, only the spots containing CaMV35S promoter probe, but not the lectin gene, were detected, indicating the high specificity of the exemplary assay described herein. Although the signal intensity decreases as the concentration of target sequence is lowered, as little as about 0.1 fmol of target may be detected for the probe spotted at a high concentration of about 1 μM. The signals may be detected at the probe concentration of about 0.01 μM, if about 100 fmol of the target PCR product is used in the assay. Spotting of probes from stock solutions higher than about 1 μM did not increase detection sensitivity in the range of target concentrations tested, thus requiring the adoption of about 1 μM probe concentration for subsequent spotting trials.

The effects of spot size were then tested, which in part may determine the number and density of spots per chip, as well as the target DNA concentration on the sensitivity of probe detection as shown in the photograph of FIG. 2b. As shown in the left panel of the photographs of FIG. 2b, biosensor chips spotted by a computer-controlled nanoliter dispenser commercially available as BioDot dispense arrayer AD3200 with 40 nl per spot at a concentration of 1.0 μM were employed. Each probe was spotted three times. The probes were then divided into three groups according to the characteristics of the target genes. The first group labeled spots 1-4 included endogenous genes for identifying plant species, including the (1) Lectin gene from soybean; (2) invertase gene (Ivrl) from maize; (3) an ACC synthase gene (Accg8) from canola, and (4) stearoyl-ACP (fiber-specific acyl carrier protein) desaturase gene (Sad1) from cotton. The second group labeled spots 5-8 included probes for detecting the promoter, selectable marker gene, and terminator of commonly used transgenes, including (5) CaMV35S promoter; (6) NOS terminator, nopaline synthase terminator (T-nos); nptII, neomycin phosphotransferase II; and, (8) GUS gene. The third group labeled spots 9-12 included probes for identifying trait genes, such as herbicide tolerance or insect resistance, including the (9) Bt toxin gene CryIAb resistant to European corn borer; (10) Bt toxin gene CryIAc resistant to lepidopteran insects; (11) the BAR gene encoding phosphinothricin acetyl transferase (pat); and, (12) a microbe-derived herbicide resistant gene from Agrobacterium strain CP4 encoding 5-enol-pyruvylshikimate-3-phosphate synthase (cp4-epsps) (See also Table 1). A representative target gene (Accg8) at the concentrations from about 0.1 to about 100 femtomoles in a 100 μl reaction was used for hybridization. Clear signals were detected when using about 1 to about 100 fmol of target DNA. Signals reached saturation at about 100 fmol according to the right panels of the photographs of FIG. 2b. The results indicated that about 10 to about 100 fmol of target DNA in a 100 μl reaction would constitute a suitable concentration range for the exemplary assay described herein. Again, no cross-reaction of target DNA at any concentration to other probes was observed, demonstrating the extraordinary specificity of the exemplary assay described herein.

Detection of Transgenes Among GM Crops Using Exemplary Thin-Film Biosensor Chips Arrays

For GMO detection, oligonucleotides were synthesized by Invitrogen (USA). PCR primers and capture probes were designed based on published sequence entries available in the GenBank database as known to one of ordinary skill in the art. The PCR primers were chosen from the following four transgenic crops: soybean, maize, canola and cotton. The four transgenic crop sequences are listed in Table 1. The reverse primers for PCR were synthesized with a biotin group at the 5′ end for detection. The 5′ termini of capture probes were modified with an aldehyde group that can react with a hydrazine group on the thin-film biosensor chip surface and followed by 10 deoxyadenosine (dA) residues as a spacer. The spacer is followed by 40 nucleotides that are complementary to the corresponding target sequence.

GMO crop seeds and negative samples of soybean, maize, canola and cotton were provided by Tianjin Customs Inspection and Quarantine Bureau of the People's Republic of China. GMO crops used here are soybean (Glycine max) of the brand Roundup Ready (RUR), Maize (Zea mays) BT11, canola (Brassica napus) RUR, and cotton (Gossypium hirsutum) SGK9708. The genomic DNA samples were isolated from seeds by the Wizard® kit (Promega, USA) according to the protocol outlined in the manual.

The genomic DNA samples from indica and japonica rice were provided by Professor Lihuang Zhu at the Institute of Genetics and Developmental Biology, Chinese Academy of Sciences. The genomic DNA sequences from Arabidopsis ecotypes of Col and Ler and two cop1 mutants were isolated from seedlings using a Qiagen DNA isolation kit. The genomic DNA samples from tomato subspecies of L. esculentum and L. pimpinellifolium were provided by Dr. Esther van der Knaap of Ohio State University.

Target DNA sequences were prepared by PCR amplification from the corresponding genomic DNA. PCR primer sets were designed using DS Gene software (Accerlrys, San Diego, Calif.) to have product sizes ranging from 110 to 350 base pairs. The PCR reactions were carried out in a 20 μl solution containing PCR reaction buffer (AmpliTaq Gold, Applied Biosystem), 2.5 mM MgCl₂, 0.2 mM dNTP, 200 nM each of forward and reverse primer, 100 ng of genomic DNA, and 1 unit of AmpliTaq Gold DNA polymerase. The PCR program used was 95° C. for 10 min, followed by 40 cycles of 94° C. for 15 sec., 56° C. for 30 sec., 72° C. for 30 sec. and final extension at 72° C. for 5 min. The PCR products were then quantified by Quant-iT Picogreen dsDNA Kits (Molecular Probe/Invitrogen) and quality was verified by electrophoresis on a 2% agarose gel.

Aldehyde-labeled oligonucleotides (probes) were spotted by manual pipette or a computer-controlled nanoliter dispenser (BioDot dispense arrayer AD3200) with 200 or 40 nl aliquots per spot, respectively, onto biosensor chips from the 1.0 μM oligonucleotide stocks in 0.1 M sodium phosphate buffer, pH 7.8. PCR amplicon targets, from 100 to 350 bp in length, at a concentration of 100 fmol each per 100 μl reaction were denatured and hybridized on the chip for 10 min. at 45° C. in hybridization buffer (5×standard saline citrate (SSC) and 5 mg ml⁻¹ acid-treated casein (ATC)). After washing three times in 0.1×SSC, the chips were incubated with an anti-biotin IgG-HRP conjugate (Jackson ImmunoResearch; 1:1,000 dilution from a 1 mg ml⁻¹ stock in a buffer containing 5×SSC/5 mg ml⁻¹ ATC/10% glycerol) for 5 min. in hybridization buffer. The chips were rinsed with 0.1×SSC three times, and then 100 μl of tetramethylbenzidine (TMB) formulation from BioFx Laboratories (Owings Mills, Md.) was added to the chips and incubated for 5 min. at room temperature. The chips were rinsed in double distilled water (ddH₂O), air-dried and scored readily by eye or photographed under a dissection microscope (Olympus, SZX12).

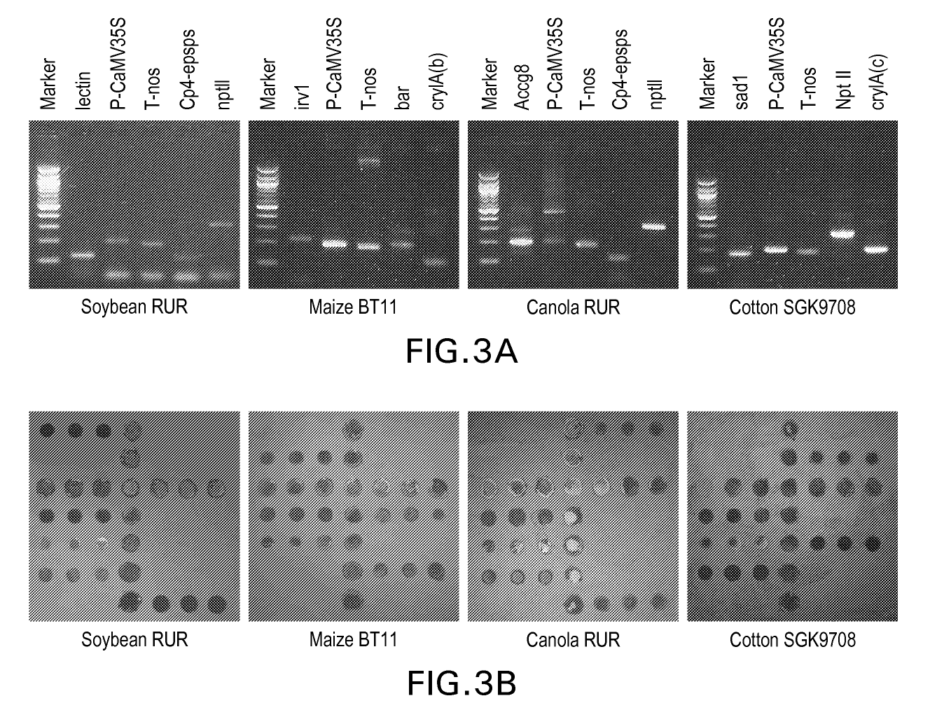

Referring again to the photograph of FIG. 2b, the biosensor chip equipped with the oligonucleotide probes shown in the left panel was used to detect transgenes from samples of genetically modified soybean, maize, canola and cotton tissues. Referring now to the photographs of FIGS. 3a and 3b, the expected DNA targets were successfully amplified by PCR and hybridized to the biosensor chip. For example, Roundup Ready soybean samples can be used to amplify five DNA targets of expected sizes corresponding to Lectin, CaMV 35S promoter, NOS terminator, cp4-epsps, and nptII. An equal volume mixture of these five PCR DNA fragment targets was used for hybridization in a 100 μl reaction. The exemplary assay described herein resulted in the detection of five sets of colored dots as shown in the bottom left corner (See FIG. 3b). Five expected DNA targets were also amplified from a maize Bt11 sample. The targets included Ivrl, CaMV 35S promoter, NOS terminator, BAR (pat) and CryIA(b) (See FIG. 3a), which were applied to the biosensor chip in an equal volume mixture containing all five sequences (See FIG. 3b). Very similar results were obtained for Roundup Ready canola samples and Bt cotton SGK9708 samples (See FIGS. 3a and 3b). In each case, all five probes hybridized to expected DNA targets and gave rise to positive identification. No false positives were observed among these tests. In all four cases, DNA targets from non-transgenic control samples only gave products corresponding to the endogenous gene in the chip assay results. These experimental results indicate the exemplary thin-film biosensor chip assay described herein may utilize one custom-designed chip to detect the presence of these four GMOs. Furthermore, the exemplary thin-film biosensor chip assay described herein may be further modified to accommodate a variety of commercialized GMO detection needs.

Detection of Indica and Japonica-Specific SNPs Using the Exemplary Thin-Film Biosensor Chip SNP Array

For each SNP, one pair of 50-nucleotide P1 capture probes were synthesized with only one base difference in their 3′ terminal nucleotide sequence corresponding to different SNP forms. The 5′ terminal nucleotide was modified for reactivity with the chip surface, and followed by an additional 10-dA residue spacer and then a 40-nucleotide sequence complementary to the corresponding target sequence. A second oligonucleotide probe (biotin-P2) contains a 20-nucleotide sequence immediately adjacent to the SNP nucleotide (FIG. 1C). Its 3′ terminus was modified with biotin for detection, while its 5′ terminus was modified with a phosphate for allele-specific ligation. The P1 and P2 probes were synthesized by Invitrogen at a 50 nmole scale without post-synthesis purification. The detailed sequences for oligonucleotide probes P1 and P2 are provided below in Table 2.

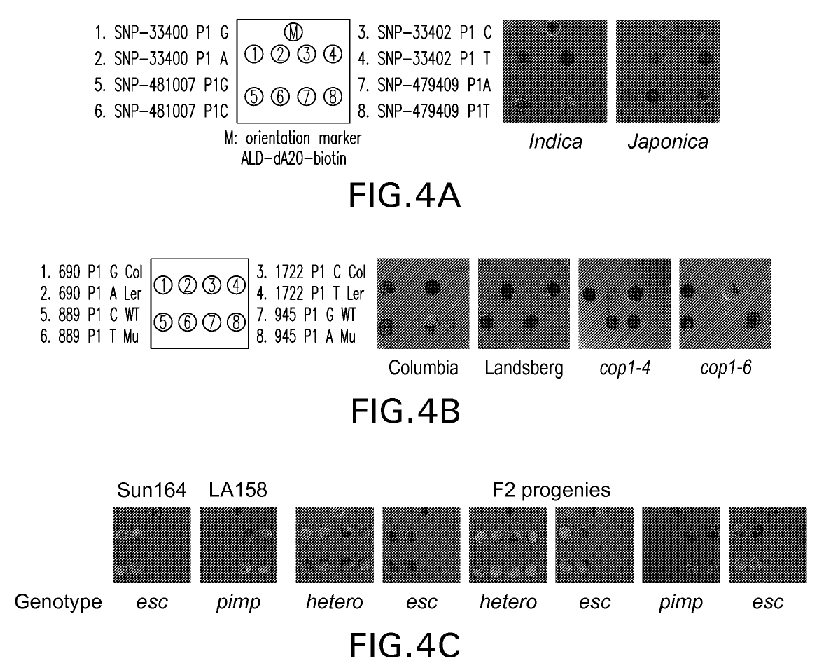

P1 capture probes were manually spotted in a format as shown in FIG. 4 on hydrazine-activated thin-film biosensor chips, commercially available from ThermoBioStar located in Louisville, Colo., from a mixture consisting of 0.2 μl of 1 μM P1 in 0.1 M phosphate buffer, pH 7.8 with 10% glycerol. After incubation for two hours at room temperature in a humid chamber, the chips were washed with 0.1% SDS and ddH₂O, and then dried. A ligation mixture, containing 20 mM Tris-HCl, pH 8.3, 25 mM KC1, 10 mM MgCl₂, 0.5 mM nicotinamide adenine dinucleotide, 0.01% Triton X-100, 5 mg ml⁻¹ acid-treated casein, 10 nM P2-biotin probes and 0.04 unit μl⁻¹ mutant Ampligase (Lys294Arg of T. thermophillus ligase), was applied to each chip and pre-warmed to 60° C. The PCR amplicons from plant samples at a concentration of about 100 fmol in 10 μl of ddH₂O were denatured at 95° C. for 3 minutes. After denaturation, 10 μl of this solution was immediately added into the pre-incubated ligation mixture and incubated at 60° C. for 10 minutes. A stringent wash with 0.01 M NaOH at 60° C. was applied three times followed by three brief rinses with 0.1×SSC (standard saline citrate) at room temperature. The chips were then incubated at room temperature for 5 minutes with 100 μl of an anti-biotin IgG-HRP conjugate, prepared by the Jackson ImmunoResearch Lab, 1:1000 dilution from a 1 mg ml⁻¹ stock in a buffer containing 5×SSC and 5 mg ml⁻¹ acid treated casein. After three brief washes with 0.1×SSC, 100 μl of a tetramethyl benzidine formulation from BioFX Lab was applied to each chip and incubated for 5 minutes at room temperature, then rinsed in ddH₂O and air dried. SNP genotypes were determined with the naked eye and the results were recorded using a dissection microscope fitted with a digital camera.

The exemplary thin-film biosensor chip assay was then tested to discriminate between indica and japonica subspecies of rice based on genome-specific SNPs, four SNP markers were selected from the indica/japonica SNP database at http://plantgenome.agtec.uga.edu/snp. These four SNPs have SNP identification numbers 33400 G/A (indica versus japonica), 33402 C/T, 481007 G/C, and 479709 A/T. Genomic DNA was isolated from two rice subspecies (9311, a cultivar of indica, and Nipponbare, a cultivar of japonica) whose complete genome sequences are available. SNP-specific target sequences were amplified by PCR with specific primer pairs in a multiplexed PCR reaction that included four sets of primers together. Referring now to the photographs of FIGS. 4a-4c and Table 2 below, the four selected SNP P1 capture probe pairs were manually arrayed on the thin-film biosensor chips with indica probes on the left and japonica probes on the right as shown in the left panel of the photographs of FIG. 4a. When PCR amplicons from indica or japonica genomic DNA were applied to the chip, signals were generated only on the four spots corresponding to the correct genome-specific SNP P1 capture probes as shown in the right panel of the photographs of FIG. 4a. A very weak cross-reaction of the two Indica specific SNP probes with the Japonica DNA targets was observed at spots 1 and 5. This cross-reaction may be easily eliminated by increasing the stringency of hybridization and washing as known to one of ordinary skill in the art. Once a PCR-amplified DNA target is obtained, the entire procedure takes about 30 minutes and may be performed using the simplest laboratory equipment, such as a 60° C. water bath for incubation and a heating block at 95° C. for denaturing DNA as known to one of ordinary skill in the art. Again, the results are visible to the unaided human eye.

The exemplary SNP assay platform on the thin-film biosensor chips may also be used to identify Arabidopsis ecotypes of Columbia (Col) and Landsberg erecta (Ler), and detect specific single nucleotide point mutations in a gene of interest. As an example, detection of SNPs and point mutations in Arabidopsis COP1 genes were carried out (See FIG. 4b). Spotting positions of capture probes from two ecotype-specific SNPs (1-4) and two point mutations in the COP1 gene (5-6) are shown on the left panel. The right panels show representative images of detection results using PCR targets from Col, Let, cop1-4 and cop1-6 as indicated. The COP1 gene, (constitutive photomorphogenic locus 1), encodes an essential regulatory protein that plays a role in the photomorphogenic development in Arabidopsis. The COP1 gene is located in the middle of the lower arm of chromosome 2, and several ecotype-specific SNP and point mutations have been reported (McNellis, T. W., et al. “Overexpression of Arabidopsis COP1 results in partial suppression of light-mediated development: evidence for a light-inactivable repressor of photomorphogenesis,” Plant Cell, 6, 1391-1400 (1994), which is incorporated by reference herein in its entirety). A procedure was optimized to detect the two ecotype SNPs at 690 G/A and 1722 C/T (Col versus Ler, number counted from the A base of COP1 gene start codon), and two cop1 mutations, cop1-4 at 889 C/T (Col wild type versus mutant) and cop1-6 at 945 G/A. Four pairs of the SNP P1 capture probes were manually spotted on the thin-film biosensor chip surface in a pattern shown in the left panel of the photographs of FIG. 4b. Target DNA samples from Col, Ler, cop1-4 and cop1-6 mutants were prepared by PCR to produce a single 1.1 kb fragment covering all of the 4 COP1 SNPs. In all cases, the DNA target always reacted with the matching P1 probes and gave rise to definitive identification of all four SNP markers as shown in the right panel of FIG. 4b.

Exemplary Thin-Film Biosensor Chip SNP Assay for Use in Marker-Assisted Breeding in Tomato

Referring again to the photographs of FIGS. 4a-4c, an exemplary assay for monitoring hereditary transmission of molecular markers in a tomato breeding was also demonstrated using the thin-film biosensor chip SNP detection platform. Two tightly linked tomato SNP markers, TG576 and BAC33R, were selected for a sample genotyping analysis in the F2 progeny (See FIG. 4c). As shown in the left panel of the photographs of FIG. 4c, the P1 capture probes were manually spotted in duplicate at the top row with TG576 P1-G (left) for the genotype of L. esculentum and TG576 P1-T (right) for genotype of L. pimpinellifolium. On the bottom row, BAC33R P1 probes were spotted in duplicate at the left for L. esculentum (P1-A) and at the right for L. pimpinellifolium (P1-G). Multiplex PCR products for both SNP target DNA regions from each of the two parents of Sun 1642 (L. esculentum) and LA1589 (L. pimpinellifolium), and representative F2 progenies were applied to the silicon chips for genotyping. Representative images of the genotypes of the two parents and six F2 progenies for both SNP markers are shown in the photograph of FIG. 4c. This exemplary SNP assay definitively determined the genotype of all F2 progeny individuals at these two SNP positions.

TABLE 2
Oligonucleotide sequences of P1, P2 probes and PCR primers for SNP detection

| | | |
|---|---|---|
| A. Rice | | |
| SNP-479409 | P1-A indica: ALD-aaaaaaaaaactacaaggagacaaataaacaaggtgttgtatataacaaa | PCR |
| at Chromosome | P1-T japonica: ALD-aaaaaaaaaactacaaggagacaaataaacaaggtgttgtatataacaat | product: |
| 10 position | P2: 5′-phosphate-aataaacacatacctggata-biotin-3′ | 150 bp |
| 16,406,758 | Primer forward: tgccgagcctgtgtattatg | |
| | Primer reverse: gctaaactattttgtgtccccc | |
| SNP-33400 | P1-G indica: ALD-aaaaaaaaaaaaagaaatttgacctatctaccataaacaattccaaagcg | PCR |
| at Chromosome | P1-A japonica: ALD-aaaaaaaaaaaaagaaatttgacctatctaccataaacaattccaaagca | product: |
| 4 position | P2: 5′-phosphate-cttcgccgcaacaaatatct-biotin-3′ | 199 bp |
| 27,582,119 | Primer forward: ggttgcatcatctgtatctcag | |
| | Primer reverse: cagcatcaatgacattaaccac | |
| SNP-33402 | P1-C indica: ALD-aaaaaaaaaacaactgtgtagctttatatagcagaaaaatatagtaaagc | PCR |
| at Chromosome | P1-T japonica: ALD-aaaaaaaaaacaactgtgtagctttatatagcagaaaaatatagtaaagt | product: |
| 4 position | P2: 5′-phosphate-gcatatagaattgaaataat-biotin-3′ | 126 bp |
| 27,582,637 | Primer forward: gcagaataggatcatgagtagc | |
| | Primer reverse: agtcctggatctgttcaaaatc | |
| SNP-481007 | P1-G indica: ALD-aaaaaaaaaaaccaggaagctccatttcttgcagaaatcagcatggaatg | PCR |
| at Chromosome | P1-C japonica: ALD-aaaaaaaaaaaccaggaagctccatttcttgcagaaatcagcatggaatc | product: |
| 4 position | P2: 5′-phosphate-tgagcctttagcatcaagtt-biotin-3′ | 161 bp |
| 27,385,971 | Primer forward: cagccaaggttggtagttc | |
| | Primer reverse: agtaggccatacaccatgatac | |

The genomic locations of four rice SNP markers are mapped to the current version (4.0) of
japonica assembly at www.TIGR.org.

| | | |
|---|---|---|
| B. Arabidopsis | | |
| SNP COP1 | P1-G Columbia: ALD-aaaaaaaaaaaatagatttataccgagctagggacagatattctgtatag | |
| position 690 | P1-A Landsberg: ALD-aaaaaaaaaaaatagatttataccgagctagggacagatattctgtataa | |
| | P2: 5′-phosphate-ttgcggatgctcggagatga-biotin-3′ | |
| SNP COP1 | P1-C Columbia: ALD-aaaaaaaaaaacacaagaaagcagtttcctatgttaaatttttgtccaac | |
| position | P1-T Landsberg: ALD-aaaaaaaaaaacacaagaaagcagtttcctatgttaaatttttgtccaat | |
| 1722 | P2: 5′-phosphate-aacgagctcgcttctgcgtc-biotin-3′ | |
| cop1-4 | P1-C wild type: ALD-aaaaaaaaaagggctaccaaagaaggatgcgctgagtgggtcagattcgc | |
| mutation | P1-T mutant in cop1-4: ALD-aaaaaaaaaagggctaccaaagaaggatgcgctgagtgggtcagattcgt | |
| | P2 5′-phosphate-aaagtttgaatcagtcaact-biotin-3′ | |
| cop1-6 | P1-G wild type: ALD-aaaaaaaaaagcacagattgcctaattctgttaaagtgtcttgtcttgtg | |
| mutation | P1-A mutant in cop1-6 ALD-aaaaaaaaaagcacagattgcctaattctgttaaagtgtcttgtcttgta | |
| | P2: 5′-phosphate-gttcaatgatttacaagaat-biotin-3′ | |
| PCR primers | Forward: 5′-tgccgttgagagacatagaatag-3′ | PCR |
| | Reverse: 5′-gtgtgctatctgtggacgcag-3′ | product: |
| | | 1123 bp |
| C. Tomato | | |
| SNP TG576 | P1-G L. esculentum: ALD-aaaaaaaaaatgaccaggttctatctctctcattctctttctttgatgtg | PCR |
| | P1-T L. pimpinellifolium: ALD-aaaaaaaaaatgaccaggttctatctctctcattctctttctttgatgtt | product: |
| | P2: 5′-phosphate-ctggttattgtttctgaaac-biotin-3′ | 150 bp |
| | Primer forward: tcatcacttggatggtaatgc | |
| | Primer reverse: tgaaactaggcagaaaagcag | |
| SNP | P1-A L. esculentum: ALD-aaaaaaaaaaaaattttaaattttgaatccgcgagcataaataatgtcga | PCR |
| BAC33R | P1-G L. pimpinellifolium: ALD-aaaaaaaaaaaaattttaaattttgaatccgcgagcataaataatgtcgg | product: |
| | P2: 5′-phosphate-agagtgatatgtgttacaac-biotin-3′ | 133 bp |
| | Primer forward: aaaacattaactacttcatccg | |
| | Primer reverse: ttttccccagaggagagtac | |

Referring now to the photographs of FIG. 4c and photographs in the top panels of FIG. 5, the exemplary assay was also used to quantify the relative abundance of SNP alleles in a mixture of DNA targets and, more specifically, to quantify target molecule abundance by measuring the signal intensity correlative with the concentration of the DNA targets. The photograph of FIG. 4c shows the SNP genotype images of the two parents and six individuals of the F2 progeny in a tomato breeding program. FIG. 5 illustrates the mapping process and collection of segregating mutant individuals in the F2 population. The mutant collection from the F2 population is pooled for DNA isolation and segregation analysis of genome-wide SNP markers. The graph illustrates the frequency of a SNP marker found on chromosome 2 conferring ecotype specificity (Col or Ler). When approaching the COP1 locus, the SNP marker is biased toward that of the parent with the mutation due to the pooled F2 mutants DNA used.

DNA fragment targets from DNA samples taken from two rice subspecies that cover SNP-470409 were PCR-amplified and artificially mixed them at different ratios (0.1:100 to 100:0.1 femtomoles). Rice SNP-479409 capture-probes P1-A (top, indica) and P1-T (bottom, japonica) were spotted by hand on chips in four replicates (200 nl per spot) using 1 μM stock solution. A mixture of the two PCR products for the SNP-479409 from indica and japonica rice within the above indicated range of ratios was applied to the exemplary thin-film biosensor chip SNP assays. As shown in the top panels of FIG. 5, direct inspection of the signal intensities indicate a strict correlation between signal intensity and target concentration, and a complete absence of cross-reaction. As shown in the lower panels of FIG. 5, the signal intensity was quantified by scanning the images and plotting the relative intensity of different samples on a graph. The exemplary biosensor chip-based SNP assay quantified the respective abundance of the two SNP populations to within a range of 100-fold (1/100 or 100/1) for the two allele-specific DNA fragment targets. The results establish the exemplary thin-film biosensor chip SNP assay is capable of detecting the presence of one specific sequence in 100 in the population in real sample analysis.

The thin-film biosensor chip assay described herein is superior to the other available assays in terms of cost, sensitivity, ease-of-use, and time required. This assay is extremely robust and economical, exhibits high sensitivity and specificity, and can be adjusted for low to high throughput applications. This technology can be customized for any nucleotide-sequence-based identification assay and is suitable for wide use in crop breeding, trait mapping, and other work requiring positive detection of specific nucleotide sequences. The assay is inexpensive due to low cost of chemical and biological reagents and the small size of biosensor chips. Furthermore, this assay is extremely robust, exhibits high sensitivity and exquisite specificity, can be adapted for low, medium, or high throughput, and most importantly, is significant less expensive compared to other high throughput alternatives. This technology can also be customized for any specialized assay based on unique nucleotide sequences. The exemplary assay described herein can thus be adapted for a variety of applications including laboratory research (such as mapping mutations), plant product inspection, marker-assisted breeding, SNP marker-based plant strain identification, and pathogen detection.

For example, the detection of microbial pathogens in plant product shipments can be done using essentially the same process as GMO detection described herein since again the parameter being measured is the gene sequence of the plant DNA sample. Various types of pathogens can be consistently detected by their presence of unique toxicity or pathogenecity related genes, such as genes encoding toxins. Thus, the presence of those pathogenic bacteria can be assayed by the presence of their respective unique DNA sequences relating to distinct toxicity or pathogenecity.

In another example, the exemplary assay described herein can be adapted easily to screen for the presence of harmful pathogens in food products, as long as some unique genomic sequence information for the pathogens in question is available. Moreover, due to its ease of use, low cost, and basic equipment requirements, this assay can be carried out by breeding research stations in developing countries that cannot afford expensive instrumentation associated with other available SNP assays. Noticeably, the thin-film biosensor chip assay described here has many advantages over gel electrophoresis of gene-specific PCR fragments in several respects. The exemplary biosensor chip assay is far more sensitive (at least a thousand fold) than gel electrophoresis, and therefore is able to detect the transgenes with small amount of material. Moreover, the exemplary assay described herein also filters out non-specific PCR amplifications, which would otherwise give rise to a false positive in gel electrophoresis and real-time PCR methods.

One or more embodiments of the present invention have been described. Nevertheless, it will be understood that various modifications may be made without departing from the spirit and scope of the invention. Accordingly, other embodiments are within the scope of the following claims.

What is claimed is:

1. A process for detecting at least one DNA sequence using an optical thin-film biosensor chip, comprising:
placing a sample to be tested in contact with at least one capture probe attached to an optical thin-film biosensor chip;
incubating said sample in the presence of an enzyme and a substrate;
identifying a change in the color of said sample; and
detecting said sample comprises at least one DNA sequence.

2. The process of claim 1, wherein identifying comprises visualizing.

3. The process of claim 1, wherein identifying comprises producing a precipitate.

4. The process of claim 3, wherein producing comprises the steps of:
binding said enzyme to said substrate in the presence of said at least one DNA sequence matching said at least one capture probe; and
converting said substrate into a precipitable product.

5. The process of claim 1, wherein said at least one capture probe comprises an oligonucleotide probe having a nucleotide sequence that matches at least a portion of said at least one DNA sequence.

6. The process of claim 1, wherein placing said sample comprises the steps of:
hybridizing said at least one DNA sequence to said at least one capture probe in the presence of a ligase using a biotinylated polymerase chain reaction; and
washing said at least one DNA sequence attached to said at least one capture probe.

7. The process of claim 6, wherein said ligase comprises a thermostable DNA ligase.

8. The process of claim 1, further comprising the step of arraying and covalently attaching at least one capture probe to an optical thin-film biosensor chip prior to placing said sample in contact with said at least one capture probe.

9. The process of claim 8, wherein arraying and covalently attaching comprises the steps of:
arraying said at least one capture probe; and
covalently attaching said at least one capture probe to said optical thin-film biosensor chip.

10. The process of claim 9, wherein said at least one capture probe includes a forward strand sequence matching at least a portion of said at least one DNA sequence and a terminus covalently attached to a surface of said optical thin-film biosensor chip.

11. The process of claim 9, wherein said optical thin-film biosensor chip comprises a surface derivatized to form covalent bonds.

12. The process of claim 1, wherein said enzyme comprises an enzyme conjugate capable of detecting said at least one DNA sequence hybridized with said at least one capture probe.

13. The process of claim 1, wherein said substrate comprises a substrate capable of binding with said enzyme in the presence of at least one DNA sequence hybridized with said at least one capture probe.

14. The process of claim 1, wherein incubating comprises incubating said sample in the presence of said enzyme and said substrate for a period of time of about 5 minutes to about 30 minutes.

15. The process of claim 1, wherein said at least one DNA sequence is a transgene.

16. The process of claim 1, further comprising detecting the presence of a pathogen in said sample, said sample comprising a genetically modified crop, based upon the detection of said at least one DNA sequence.

17. The process of claim 1, further comprising mapping at least one trait of said sample, said sample comprising a genetically modified crop, based upon the detection of said at least one DNA sequence.

18. The process of claim 1, further comprising detecting at least one pathogen or at least one toxin in said sample, said sample comprising a genetically modified crop, based upon the detection of said at least one DNA sequence.

19. A process for detecting at least one SNP using an optical thin-film biosensor chip, comprising:
placing a sample to be tested in contact with at least one capture probe attached to an optical thin-film biosensor chip;
incubating said sample in the presence of an enzyme and a substrate;
identifying a change in the color of said sample; and
detecting said sample comprises at least one SNP.

20. The process of claim 19, wherein identifying comprises visualizing.

21. The process of claim 19, wherein detecting comprises producing a precipitate.

22. The process of claim 21, wherein producing comprises the steps of:
binding said enzyme to said substrate in the presence of said at least one SNP assay matching said at least one capture probe; and
converting said substrate into a precipitable product.

23. The process of claim 22, wherein said at least one capture probe comprises a first oligonucleotide probe and second oligonucleotide probe.

24. The process of claim 23, wherein said first oligonucleotide probe comprises a nucleotide sequence that compliments at least a portion of one side of said at least one SNP assay and said second oligonucleotide probe comprises a nucleotide sequence that compliments at least a portion of an opposing side adjacent to said at least one SNP assay.

25. The process of claim 23, wherein said first oligonucleotide probe includes a 3′ terminus having a reactive group for detecting said at least one SNP assay and a 5′ terminus covalently attached to a surface of said optical thin-film biosensor chip.

26. The process of claim 23, wherein said second oligonucleotide probe includes a 3′ terminus having a reactive group for detecting said at least one SNP assay and a 5′ terminus covalently attached to a surface of said optical thin-film biosensor chip.

27. The process of claim 19, further comprising the step of arraying and covalently attaching at least one capture probe to an optical thin-film biosensor chip prior to placing said sample in contact with said at least one capture probe.

28. The process of claim 27, wherein arraying and covalently attaching comprises the steps of:
arraying a first capture probe and a second capture probe;
covalently attaching said first capture probe and said second capture probe to a surface of said optical thin-film biosensor chip.

29. The process of claim 28, wherein said surface is derivatized to form covalent bonds.

30. The process of claim 19, wherein placing said sample comprises the steps of:
hybridizing said at least one SNP assay to said at least one capture probe in the presence of a ligase using a biotinylated polymerase chain reaction; and
washing said at least one SNP assay attached to said at least one capture probe.

31. The process of claim 30, wherein said ligase comprises a thermostable DNA ligase.

32. The process of claim 19, wherein said enzyme comprises an enzyme for detecting at least one SNP assay hybridized with said at least one capture probe.

33. The process of claim 19, wherein said substrate comprises a substrate for binding with said enzyme in the presence of at least one SNP assay hybridized with at least one capture probe.

34. The process of claim 19, wherein incubating comprises incubating said sample in the presence of said enzyme and said substrate for a period of time of about 10 minutes to about 30 minutes.

35. The process of claim 19, wherein said sample comprises a plant.

36. The process of claim 19, further comprising mapping a mutation in said sample, said sample comprising a plant, based upon the detection of said at least one SNP.

37. The process of claim 19, further comprising identifying a strain of said sample, said sample comprising a plant, based upon the detection of said at least one SNP.

38. The process of claim 19, further comprising detection a pathogen in said sample, said sample comprising a plant, based upon the detection of said at least one SNP.

39. The process of claim 19, further comprising mapping at least one trait of said sample, said sample comprises a plant, based upon the detection of said at least one SNP.

40. The process of claim 19, further comprising detecting at least one pathogen or at least one toxin in said sample, said sample comprising a plant, based upon the detection of said SNP.
